(19) United States
(12) Patent Application Publication
(10) Pub. No.: US 2015/0272698 A1
(43) Pub. Date: Oct. 1, 2015

(54) NAVIGATION TOOLS USING SHAPE SENSING TECHNOLOGY

(71) Applicant: Regents of the University of Minnesota, Minneapolis, MN (US)

(72) Inventors: Christopher ROLFES, St. Paul, MN (US); Douglas POST, Medford, MA (US); Alan JOHNSON, Grand Forks, ND (US); Raed RIZQ, Maple Grove, MN (US)

(73) Assignee: Regents of the University of Minnesota

(21) Appl. No.: 14/621,045

(22) Filed: Feb. 12, 2015

Related U.S. Application Data
(60) Provisional application No. 61/973,046, filed on Mar. 31, 2014.

(51) Int. Cl. A61B 19/00; A61B 5/06; A61B 5/00

(52) U.S. Cl. CPC A61B 19/5244; A61B 5/0084; A61B 5/6847; A61B 5/066; A61B 2019/5261

(57) ABSTRACT

A navigation system for an airway of a lung includes a wire configured to be inserted into the airway of a patient. The wire may include at least one fiber optic cable with a bragg grating. The system may also include a processing system configured to display a map of at least a portion of the airway. The processing system may be operably coupled to the wire and may be configured to identify a location of the wire on the map.

### CROSS-REFERENCE TO RELATED APPLICATIONS

This application claims the benefit of priority from U.S. Provisional Application No. 61/973,046, filed on Mar. 31, 2014, the entirety of which is incorporated herein by reference.

### TECHNICAL FIELD

The disclosed embodiments relate to navigation tools and methods of navigation using shape sensing technology. More particularly, the present disclosure relates to devices and methods of lung navigation using a smart wire with shape sensing technology.

### BACKGROUND

A biopsy is a medical procedure performed to remove tissue or cells from the body for examination. A lung biopsy is a procedure in which samples of lung tissue are removed from a nodule or a portion of the lung to determine if lung disease or cancer is present. A lung biopsy may be performed using either a closed or an open method. An open biopsy is performed in the operating room under general anesthesia and typically requires hospitalization. Closed biopsy is performed through the skin or through the trachea of the patient. Several types of closed biopsy methods (e.g., needle biopsy and transbronchial biopsy) are known. In needle biopsy, under local anesthesia, the doctor uses a needle that is guided through the chest wall to a desired area of the lung under computed tomography (CT) or fluoroscopy to obtain a tissue sample. This type of biopsy (sometimes referred to as transthoracic or percutaneous biopsy) is typically used to remove tissue from nodules located at the peripheral regions of the lung (e.g., close to the rib cage). Known risks of needle biopsy include pneumothorax and excessive bleeding.

Transbronchial biopsy is performed using a catheter (e.g., endoscope, bronchoscope, etc.) inserted through the main airways of the lungs. The catheter may be directed to a desired area of the lung airway using imaging techniques such as endobronchial ultrasound. Various types of biopsy tools may be inserted through the catheter to obtain lung tissue for examination. Nodules within the first two branches of the lung airways can typically be accessed using this method. While transbronchial biopsy is generally considered to be lower risk than needle biopsy, because of limitations of the catheter and/or imaging techniques, this technique can typically access nodules in only the larger branches of the airway and therefor it is only used in a subset of patients who need biopsy.

There is considerable interest in lung navigation techniques that increase the volume of the lung that can be accessed using transbronchial biopsy techniques. Some of techniques use electromagnetic (EM) navigation in which an EM field is created and used to track the three dimensional location and orientation of the tip of an inserted catheter. A reconstructed 3D scan (e.g., CT, MRI, etc.) of the patient is aligned with the physical patient position using a best fit algorithm. As the catheter is moved through the lung, the position is projected in the scan, allowing the doctor to navigate within the lung. The disadvantages of this approach include the complexities involved in setting up the EM fields, and the challenges involved in using certain medical technologies (magnetic resonance imaging, radiofrequency ablation, microwave ablation, etc.) in combination with this technique. The systems and methods of the current disclosure may alleviate some of the limitations discussed above.

### SUMMARY

In one aspect, a navigation system for an airway of a lung is disclosed. The system may include a wire configured to be inserted into the airway of a patient, wherein the wire includes at least one fiber optic cable with a bragg grating. The system may also include a processing system configured to display a map of at least a portion of the airway. Wherein the processing system is operably coupled to the wire and is configured to identify a location of the wire on the map.

Additionally or alternatively, in some aspects, the system may include one or more of the following features: the wire may include at least two fiber optic cables positioned within a flexible tube, each fiber optic cable including a bragg grating; the wire may include at least three fiber optic cables positioned within a flexible tube, each fiber optic cable including a bragg grating; the wire may include four fiber optic cables positioned within a flexible tube, each fiber optic cable including a bragg grating; the four fiber optic cables may be arranged symmetrically about a longitudinal axis of the flexible tube; one or more keys may serve as reference points to identify the wire in the map; a catheter configured to be inserted into the airway, wherein the wire extends into the airway through the catheter, and wherein the wire includes one or more markers configured to identify a length of the wire extending out of the catheter on the map; a tool extending into the airway through the catheter, wherein a portion of the wire extends through the tool into the airway; a distal end of the wire may include an image sensor; and a distal end of the wire may include one or more electrodes.

In another aspect, a navigation system for an airway of a lung is disclosed. The system may include a wire configured to be inserted into the airway of a patient. The wire may include at least three fiber optic cables extending longitudinally through a flexible tube. Each fiber optic cable may include a bragg grating. The system may also include a tool coupled to the wire and configured to extend into the airway, and a processing system configured to display a map of at least a portion of the airway. The processing system may be operably coupled to the wire and may be configured to identify a location of the wire on the map.

Additionally or alternatively, in some aspects, the system may include one or more of the following features: the at least three fiber optic cables may be configured to slide relative to one another within the flexible tube, and the processing system may be configured to detect a curvature of each of the at least three fiber optic cables; the system may include an imaging device coupled to a distal end of the wire; one or more keys that serve as reference points to identify the wire on the map.

In a further aspect, a method of navigating an airway of a lung is disclosed. The method may include inserting a wire into the airway. The wire may include at least one fiber optic cable with a bragg grating. The method may also include tracking a location of the wire on a digital reconstruction of at least a portion of the airway.

Additionally or alternatively, in some aspects, the method may include one or more of the following steps: inserting the wire may include inserting a catheter into the airway and inserting the wire into the airway through the catheter; tracking a location includes tracking a length of the wire that extends into the airway out of the catheter; inserting a tool into the airway with the tool tracking the wire; inserting the tool may include extending a portion of the wire through the tool; inserting a wire may include inserting a wire that includes three or more fiber optic cables with bragg gratings into the airway; and tracking a location may include calculating a curvature of the wire in the airway.

### BRIEF DESCRIPTION OF THE DRAWINGS

The drawings illustrate the design and utility of exemplary embodiments of the present disclosure, in which similar elements are referred to by common reference numerals. In order to better appreciate how the above-disclosed and other advantages and objects of the present disclosure are obtained, a more detailed description of the present embodiments will be rendered by reference to the accompanying drawings. Understanding that these drawings depict only exemplary embodiments of the disclosure and are not therefore to be considered limiting in scope, the disclosure will be described and explained with additional specificity and detail through the use of the accompanying drawings in which:

FIG. 1 is a schematic illustration of an exemplary smart wire navigation system;

FIGS. 2A-2F illustrate cross-sectional views of exemplary smart wires used in the system of FIG. 1;

FIG. 3 illustrates a cross-sectional view of another exemplary smart wire used in the system of FIG. 1;

FIG. 4 illustrates a schematic view of a distal portion of an exemplary smart wire used in the system of FIG. 1;

FIG. 5A illustrates another exemplary embodiment of a smart wire navigation system of the current disclosure; and

FIG. 5B illustrates another exemplary embodiment of a smart wire navigation system of the current disclosure.

### DETAILED DESCRIPTION OF EXEMPLARY EMBODIMENTS

The present disclosure is drawn to devices and methods for navigation inside a human body. Exemplary embodiments are drawn to devices and methods for lung navigation using shape sensing technology. While the principles of the present disclosure are described with reference to lung navigation, it should be understood that the disclosure is not limited thereto. Rather, the devices and methods of the current disclosure may be used for the navigation of any internal passageway of a body.

In a human body, a wind pipe or trachea connects the nose and mouth to the lungs. As the individual inhales, air flows into the lungs through the nose and mouth. The trachea divides into the left and right bronchus stems, which further divide into progressively smaller bronchi (primary, secondary, and tertiary bronchi) and bronchioles that eventually terminate in a plurality of alveoli. The alveoli are small air sacs in the lungs that enable gas exchange with the bloodstream. In this disclosure, the lumens in the lungs that transport air from the trachea to the alveoli are collectively referred to as air passages 20.

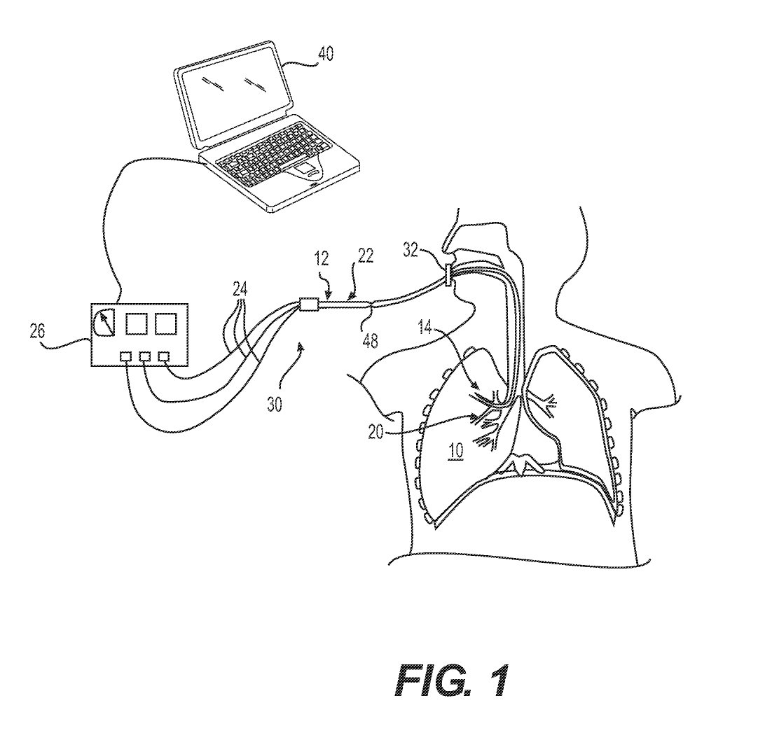

FIG. 1 is a schematic illustration of a disclosed exemplary device 30 used for lung navigation. Device 30 includes a wire 22 (or smart wire) inserted into the air passages 20 of a lung 10 through the mouth (or another orifice) of the patient. Smart wire 22 may include a flexible fiber optic cable with a proximal end 12 and distal end 14. The fiber optic cable may include a bragg grating incorporated therein. As is known in the art, bragg gratings may include periodic variations in the refractive index of the core of the fiber optic cable. Any commercially available fiber optic cable with a bragg grating may be used as smart wire 22. The proximal end 12 of the smart wire 22 may be electrically coupled to a control device 26. The control device 26 may include a tunable light source adapted to direct a beam of light through the smart wire 22 and sensing devices (or circuitry) adapted to detect the strain and curvature along the length of the smart wire 22 based on the reflected light beam. The tunable light source directs light at a selected wavelength (tuned to the specific bragg grating) through the smart wire 22. Based on the characteristics of the reflected light, the control device 26 determines the strain and curvature of the smart wire 22.

The control device 26 may direct the detected strain and curvature to a processing device 40 (such as, a computer) that calculates the location along the length of the smart wire 22 the strain/curvature was detected. For instance, in an embodiment where bragg gratings are inscribed at specific locations (for example, near the tip) of the wire 22, the processing device 40 may attribute the detected strain to that location of the bragg grating. In embodiments where bragg gratings are inscribed at multiple locations (over substantially the entire length) of the smart wire 22, the processing device 40 may use software to identify the specific location of the smart wire 22 that corresponds to the detected strain. Since strain measurement using a fiber optic cable with bragg gratings is known in the art, for the sake of brevity, this is not discussed in more detail herein. In some embodiments, the above described functions of control device 26 and processing device 40 may be performed by a single device or may be divided among a plurality of devices.

Processing device 40 may include a digital reconstruction of the patient's airway 20, and software adapted to track and display the position of the smart wire 22 in the digital reconstruction (digital map). In some embodiments, a CT scan may be used to obtain and store a digital map of the patient's airway on the processing device 40. The software may align the digital map with the actual patient position using a software component that tracks the length of the smart wire 22 positioned within the airway 20 at any time. For instance, one or more keys 32 positioned at known locations on the patient (for example, the mouth of the patient) or the smart wire 22 may provide reference points that may be used to correlate and orient the digital map to the actual patient location. As the smart wire 22 is inserted into the airway 20 through the mouth, based on the location of the detected strain, the processing device 40 may continuously track the smart wire 22 position in the airway 20.

In some embodiments, a best fit algorithm may be used to assist in aligning the digital map with the patient position, and computing the position of the smart wire 22 in the digital map. In some embodiments, an active registration method may be used to adjust the alignment and/or correct the computed position of the smart wire 22. For instance, when the computed position of the smart wire 22 is outside a predicted airway 20 location, a weighting factor that depends on the position history (for example, the current and immediately preceding position) of the smart wire 22 may be used to correct this discrepancy. Active registration may assist in realigning the digital map with the patient position and correct for uncontrollable patient movements (for example, breathing, coughing, migration of the endotracheal tube, head movement, etc.).

Although FIG. 1 illustrates a single key 32 positioned at the mouth of the patient, in general, one or more keys 32 may be positioned at any location to assist in alignment. Positioning a key 32 proximate the entrance to the airway 20 (trachea or the mouth) may help in identifying the portion of the smart wire 22 within the airway 20, and thus improve registration and alignment. Positioning a key 32 at the mouth (or trachea) may help in differentiating between the portions of the smart wire 22 that are inside and outside the patient. Further, by not including the portion of the smart wire 22 outside the patient in computations, computational time may be saved and alignment improved.

The key 32 may be integrated into or attached to the smart wire 22 (or to a guide tube or an endotracheal tube through which the smart wire 22 is deployed). The key 32 may be a structure having any geometric shape that is recognized by the software. In some embodiments, one or more electrodes may serve as the key 32. During application, these electrodes may contact the smart wire 22, and send signals that serve as reference points. In some embodiments, key 32 may include a light source detectable by the control or processing device 26, 40.

Other methods to align the physical patient position to the digital map may be used instead of, or in conjunction with, the key 32. For instance, locations of the patient's body may be used to align to the body surface to the digital map. For instance, one or more reference points on the patient's body may be correlated to locations on the digital map and used to align the digital map with the patient position. In some embodiments, a second fiber may be positioned at a known location on the patient's body (e.g., chest, along the sternum, etc.) and signals from the second fiber (light, etc.) may be directed to the processing device 40 to correlate the location of the second fiber on the digital map and align the digital map to the patient.

In some embodiments, back scatter x-ray and/or millimeter wave scanners may be used to align the digital map to the actual patient position. These techniques are commonly used in security screening to penetrate clothing and to image body surface contours. Since these imaging techniques are known in the art, they are not discussed herein. The body contours obtained by these techniques may be input into a best fit algorithm to align the digital map to the patient position.

The digital map with the location of the smart wire 22 may be displayed on a display device associated with the processing device 40. The digital map may be a two-dimensional image, such as, a photo or an x-ray, or a three or multi-dimensional map (representation of multiple spatial dimensions with an indication of change over time). A three dimensional map may assist in locating the smart wire 22 in the volume of the lung 20, and the representation of time may help compensate for patient movement (caused by breathing, etc.) Additional information that may be included in the digital map may include, for example, rotational movement of the smart wire 22, temperature at different locations on the smart wire 22, pressure at a region in the airway 20, or acceleration changes of the smart wire 22, etc.

The proximal end 12 of the smart wire 22 may be directly connected to the control device 26 or electrically coupled to the control device 26 using standard optical fibers 24 (without a bragg grating). That is, the proximal end 12 of the smart wire 22 may be coupled to standard optical fibers 24 which may then be connected to the control device 26. The connection of the smart wire 22 to the control device 26 may minimize light loss and refraction. The distal end 14 of the smart wire 22 may be inserted into the patient and pushed down the airways 20 of the lungs 10.

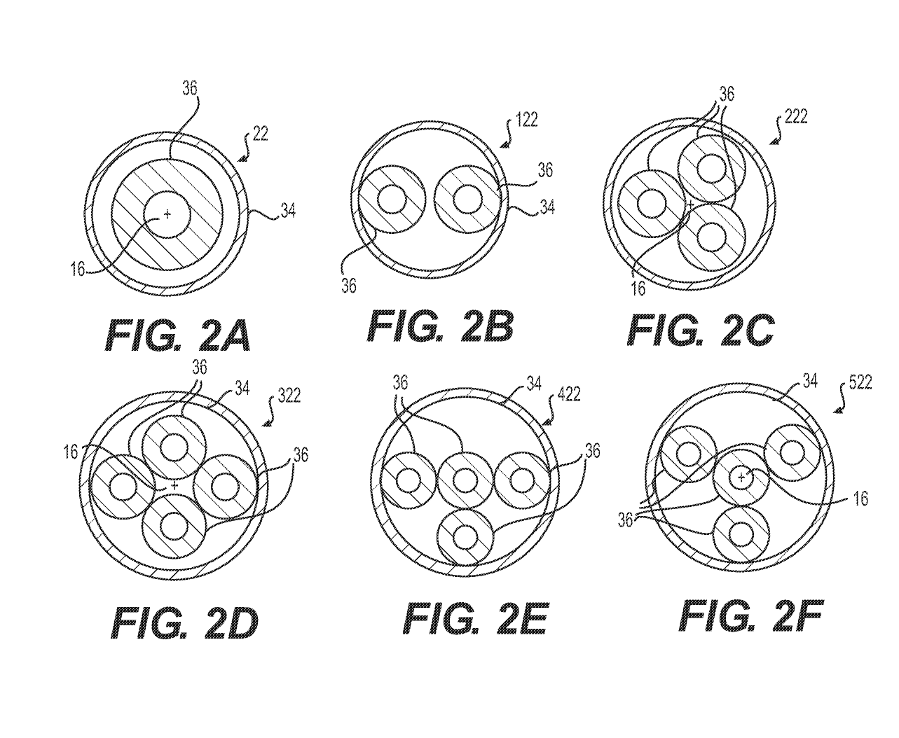

FIGS. 2A-2F illustrate cross-sectional views of exemplary smart wires of the current disclosure. The smart wire may be a bare fiber optic cable 36 or it may include one or more fiber optic cables 36 positioned within a flexible tube 34. The one or more fiber optic cables 36 may be positioned in the flexible tube 34 such that they deflect together and maintain their relative position (i.e. maintain their cross sectional orientation). The fiber optic cables 36 may not be bound to each other or to the tube 34. When the tube 34 bends or deflects, the fiber optic cables 36 within may slide relative to one another. As illustrated in FIG. 2A, in some embodiments, the smart wire 22 includes a single fiber optic cable 36 positioned in a flexible tube 34. Typically, the smart wire 22 with a single fiber optic cable 36 may be adapted to measure the curvature of the smart wire 22 in any one direction (x, y, or z). As explained below, additional fiber optic cables may be incorporated into the smart wire 22 to measure curvature in multiple directions.

In some embodiments, as illustrated in FIGS. 2B and 2C, the smart wire 122, 222 may include two or three fiber optic cables 36 within a flexible tube 32. Each fiber optic cable 36 may detect the curvature in a different direction. Therefore, the smart wire 122 of FIG. 2B may be adapted to measure the curvature in two directions, and the smart wire 222 of FIG. 2C may be adapted to measure the curvature in three directions. In some embodiments, the three fiber optic cables 36 of smart wire 222 may be arranged symmetrically about a longitudinal axis 16 of the smart wire 222 (as illustrated in FIG. 2C). The differences in curvature between the different fiber optic cables 36 may be used to track the movement of the smart wires 122, 222 in different directions.

In some embodiments, as illustrated in FIGS. 2D-2F, the smart wires 322, 422, 522 may include four fiber optic cables 36 positioned within a flexible tube 34. Three of the four fiber optic cables 36 may be used to determine curvature in the different spatial directions (x, y, and z), and the fourth fiber optic cable 36 may be used to correct for any rotational motion of the fiber (twist) based on differences in measured curvature between the fiber optic cables 36. As illustrated in FIGS. 2D-2F, the four fiber optic cables 36 may be arranged in any configuration (symmetric or non-symmetric about the longitudinal axis 16) in the flexible tube 34. For some applications, smart wires 322, 422, 522 with four fiber optic cables 36 may be preferred. It is also contemplated that, in some embodiments, smart wires 22 may include additional fiber optic cables 36 to measure additional parameters (for example, temperature, pressure, acceleration, etc.). In some embodiments, in addition to, or in lieu of, detecting curvature, some or all the fiber optic cables 36 (of FIGS. 2A-2F) may be used to measure other parameters (temperature, pressure, etc.).

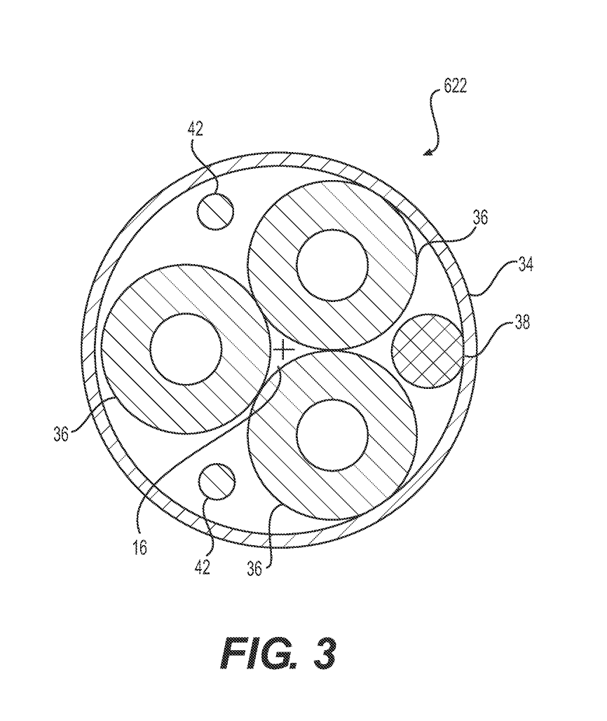

In some embodiments, smart wire 22 may include additional features. FIG. 3 illustrates a cross-sectional view of an exemplary smart wire 622 that includes a shapeable wire ribbon 38 and pull wires 42. The shapeable wire ribbon 38 may be configured to impart a desired shape to a selected region of the smart wire 622. In some embodiments, the wire ribbon 38 may be located at the distal portion of the smart wire 622. In some embodiments, the wire ribbon 38 may extend from a distal portion to a proximal portion of the smart wire 622. In some embodiments, the wire ribbon 38 may be moveable along the length of the smart wire 622. The shapeable wire ribbon 38 may allow the operator to selectively change the stiffness of various portions of the smart wire 622. The wire ribbon 38 may be made of any suitable material. In some embodiments, wire ribbon 38 may be made of an easily deformable material that holds its shape (for example, such as stainless steel). It is also contemplated that other materials such as shape memory alloys, electrically activated material combinations (bimetallic strips), etc. may be used as the wire ribbon 38.

Alternatively or additionally, smart wire 622 may include one or more pull wires 42 to assist in navigation of the smart wire 622. As is known in the art, these pull wires 42 may be coupled to an actuation mechanism (knobs, slider, etc.) at the proximal end of the smart wire 622. The operator may manipulate the actuation mechanism to selectively change the tension of one or more of the pull wires 42 and cause the distal end of the smart wire 622 to deflect. In some embodiments, the distal portion of the smart wire 622 may be configured to have a lower rigidity to allow for greater deflection of the distal portion. In some embodiments, the smart wire 622 and the pull wires 42 may be configured to limit the deflection of the proximal portion of the smart wire 622. The pull wires 42 may be coupled to the smart wire 622 in any manner. In some embodiments, the pull wires 42 may extend alongside the fiber optic cables 36 through a central lumen of the flexible tube 34, while in other embodiments, the flexible tube 34 may have lumens arranged in its wall for the pull wires 42. In some embodiments, the pull wires 42 may be fixed (for example, by welding, gluing, bonding, etc.) to the distal end of the flexible tube 34.

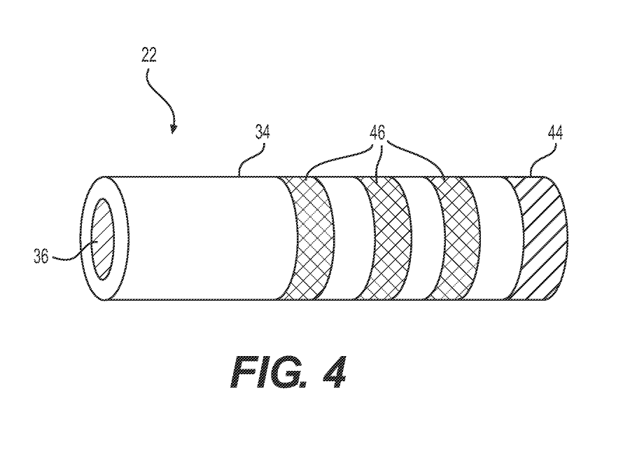

In addition to the fiber optic cables 36, the smart wire 22 may include other devices that assist in navigation. As illustrated in FIG. 4, in some embodiments, the smart wire 22 may include an imaging device 44 (and/or light sources such as, for example, LEDs) positioned at its distal end 14. Any type of image sensor (CCD, CMOS, etc.) may be used as imaging device 44. In some embodiments, imaging device 44 may have a diameter less than or equal to about 0.040 inches. The imaging device 44 may provide direct visualization to the operator to increase ease of navigation and provide visual information about the location of the smart wire 22 and the airway 20 in front to the imaging device 44.

Alternatively or additionally, the smart wire 22 may include one or more surface electrodes 46 at or near its distal end 14. In some embodiments, these electrodes 46 may be positioned on the surface of the flexible tube 34 of smart wire 22. These electrodes 46 may be electrically coupled to devices adapted to detect the electrical properties near the location of the electrodes 46. A smart wire 22 with multiple electrodes 46 may permit several measurements to be made simultaneously at different physical locations. The electrodes 46 may be coupled to software that compares readings and calculates differential readings. The differential readings may be used to locate variations in the medium that the smart wire 22 is navigating in. For example, differential electrode readings may detect the presence of a nodule in lung tissue, and the multiple electrodes 46 may help in predicting its location along the length of the smart wire 22.

After the distal end 14 of the smart wire 22 is suitably positioned in an airway 20 (for example, proximate a nodule or a lesion), surgical tools (or a catheter) may be coupled (or tethered) to the smart wire 22 and delivered to the airway 20 over the smart wire 22. The tools that may be tracked over the smart wire 22 may include, among others, biopsy forceps, biopsy needle, fiducial placement tools, ablation tools, suction catheters, etc. Since methods of delivering tools over a guide wire to a work site within a body are well known in the art (over the wire and monorail techniques, etc.), for the sake of brevity, these details are not included herein.

The smart wire 22 may have one or more mating features 48 (see FIG. 1) configured to couple tools to its surface without disconnecting the smart wire 22 from the control device 26. Any known mating feature 48 may be incorporated on the smart wire 22 for this purpose. In some embodiments, the mating feature 48 may include a slot or a groove that extends longitudinally along the surface of the smart wire 22. In such embodiments, a corresponding projection on a surgical tool may be inserted into the slot, and the tool pushed down the airway 20 over the smart wire 22. In some embodiments, the mating feature 48 may include a region of the smart wire 22 where a change or reduction in its surface cross-section enables the coupling of a tool to the smart wire 22 at that region.

It is also contemplated that, in some embodiments, rather than delivering a tool (or a catheter) to an internal work site over the smart wire 22, the smart wire 22 may be embedded on the tool or the catheter. For example, in some embodiments, a smart wire 22 may be embedded in a catheter configured to be inserted into the patient. As the catheter is inserted into a patient and navigated through tortuous air passages (or other body passages), a control system 26 may detect the change in curvature of the catheter and track its progress on a digital map of the air passage 20 (or the body passage).

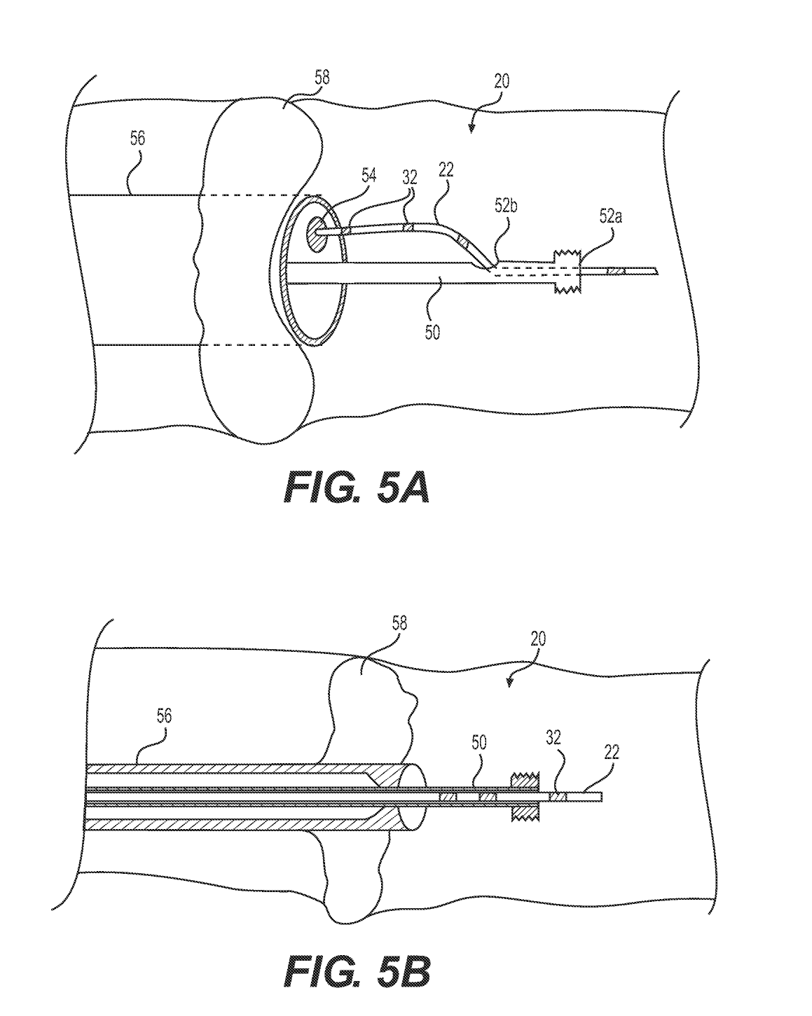

In some embodiments, as illustrated in FIG. 5A, a smart wire 22 may be introduced into an airway 20 through a catheter 56. The catheter 56 may include a lumen 54 that extends longitudinally along a length of the catheter 56. In some embodiments, lumen 54 may be configured for a specific smart wire 22 and may have one or more keys 32 along the length and/or at the distal end of the catheter. For instance, the diameter of the lumen 54 may be configured to match (for example, slightly larger than) a specific smart wire 22. A smart wire 22 may extend into the airway 20 through the lumen 54. A tool 50, preloaded on the smart wire 22, may extend into the airway 20 through the catheter 56. Tool 50 may include any type of device that assists in a desired procedure in the airway 20. The smart wire 22 may be threaded through openings (for example, through distal opening 52a and port 52b) at the distal region of the tool 50, and the tool 50 and the smart wire 22 extended proximally into the catheter 56. In some embodiments, the tool 50 and the smart wire 22 may extend into the catheter 56 through a same lumen (for example, a central lumen), while in other embodiments, the tool 50 and the smart wire 22 may extend into the catheter 56 through separate lumens. For example, as illustrated in FIG. 5A, the smart wire 22 may extend into the catheter 56 through lumen 54 and the tool 50 may extend into the catheter 56 through a central lumen.

During a medical procedure, the catheter 56 with the preloaded tool 50 and smart wire 22 may be inserted into an airway 20. At a desired location within the airway 20 (such as, for example, a primary or secondary bronchi), an inflatable balloon 58 at the distal end of the catheter 56 may be inflated to anchor the catheter 56 in the airway 20. The smart wire 22 may be pushed distally into the catheter 56 to extend into narrower regions of the airway 20 (such as, for example, a tertiary bronchi or a bronchiole) through the tool 50. The tool 50 may then be pushed into the catheter 56 to extend into these narrower regions over the smart wire 22. The control system 26 (and/or the processing device 40) may track the portion of the smart wire 22 extending out of the catheter 56 (and/or the distal end of the tool 50) into the airway 20. The smart wire 22 may include keys 32 (or other markers) along the length of the smart wire 22. The keys 32 that extend out of the catheter 56 (and/or the distal end of the tool 50) may be configured to be detected by the control device 26 (and/or the processing device 40), and may assist in determining the length of the smart wire 22 to be factored in calculations.

In some embodiments, as illustrated in FIG. 5B, the smart wire 22 and the tool 50 may be configured as an over the wire system. In some such embodiments, lumen 54 may not be used or needed, and the smart wire 22 may extend into the airway 20 through the catheter 56. The distal end of the catheter 56 may be configured with a taper and include a lubricious coating to clamp the smart wire 22 at this location and thereby minimize slack and rotation of the smart wire 22. The catheter 56 may be inserted into an airway and anchored at a location by inflating the inflatable balloon 58. The smart wire 22 may them be extended into the airway 20 by pushing the smart wire 22 into the catheter 56. Keys 32 or markers on the smart wire 22 may assist the control device 26 in determining the length of the smart wire 22 extended into the airway 20, and track the smart wire 22 on the digital map. The tool 50 may then be extended into the airway 20 over the smart wire 22.

In the embodiments of FIGS. 5A and 5B, the accuracy of tracking may be improved because only a smaller length of the smart wire 22 is used in the computations to track the location of the smart wire 22 on the digital map. In some embodiments, the catheter 56 and/or inflatable balloon 58 may also incorporate position and orientation detection capability, such as shape sensing technology, that may assist in locating and orienting the distal portion of the catheter 56, for example at the distal end of lumen 54. The larger catheter 56 may allow for more accurate shape sensing and positioning. In some embodiments, the proximal part of the smart wire 22 may be designed to primarily carry the signal from the bragg gratings in the smart wire back to the processor. This results in a shorter length of smart wire 22 to be monitored for location and rotation and thereby increase the accuracy of the system. Location and accuracy may also be improved because lumen 54 (FIG. 5A) may be designed to minimize system slack and rotation of the smart wire 22 in the proximal portion of the system.

Although a smart wire 22 configured to navigate air passages 20 of a lung 10 is described herein, it should be noted that a smart wire 22 of the current disclosure may be adapted for any purpose (such as, for example, endoscopic procedures). Moreover, while specific exemplary embodiments may have been illustrated and described herein, it should be appreciated that combinations of the above embodiments are within the scope of the disclosure. Other exemplary embodiments of the present disclosure will be apparent to those skilled in the art from consideration of the specification and practice of the exemplary embodiments disclosed herein. It is intended that the specification and examples be considered as exemplary only, and departures in form and detail may be made without departing from the scope and spirit of the present disclosure as defined by the following claims.

What is claimed is:

1. A navigation system for an airway of a lung, comprising:
a wire configured to be inserted into the airway of a patient, wherein the wire includes at least one fiber optic cable with a bragg grating; and
a processing system configured to display a map of at least a portion of the airway, wherein the processing system is operably coupled to the wire and is configured to identify a location of the wire on the map.

2. The system of claim 1, wherein the wire includes at least two fiber optic cables positioned within a flexible tube, each fiber optic cable including a bragg grating.

3. The system of claim 2, wherein the wire includes at least three fiber optic cables positioned within a flexible tube, each fiber optic cable including a bragg grating.

4. The system of claim 2, wherein the wire includes four fiber optic cables positioned within a flexible tube, each fiber optic cable including a bragg grating.

5. The system of claim 4, wherein the four fiber optic cables are arranged symmetrically about a longitudinal axis of the flexible tube.

6. The system of claim 1, wherein the system further includes one or more keys that serve as reference points to identify the wire on the map.

7. The system of claim 1, wherein the system further includes a catheter configured to be inserted into the airway, wherein the wire extends into the airway through the catheter.

8. The system of claim 7, further including a tool extending into the airway through the catheter, wherein a portion of the wire extends through the tool into the airway.

9. The system of claim 7, wherein the wire includes one or more markers configured to identify a length of the wire extending out of the catheter on the map.

10. The system of claim 1, wherein a distal end of the wire includes one or more electrodes.

11. A navigation system for an airway of a lung, comprising:
a wire configured to be inserted into the airway of a patient, wherein the wire includes at least three fiber optic cables extending longitudinally through a flexible tube, each fiber optic cable including a bragg grating;
a tool coupled to the wire and configured to extend into the airway; and
a processing system configured to display a map of at least a portion of the airway, wherein the processing system is operably coupled to the wire and is configured to identify a location of the wire on the map.

12. The system of claim 11, wherein the at least three fiber optic cables are configured to slide relative to one another within the flexible tube, and the processing system is configured to detect a curvature of each of the at least three fiber optic cables.

13. The system of claim 11, wherein the system includes an imaging device coupled to a distal end of the wire.

14. The system of claim 11, wherein the system includes one or more keys that serve as reference points to identify the wire on the map.

15. A method of navigating an airway of a lung, comprising:
inserting a wire into the airway, the wire including at least one fiber optic cable with a bragg grating; and
tracking a location of the wire on a digital reconstruction of at least a portion of the airway.

16. The method of claim 15, wherein:
inserting the wire includes inserting a catheter into the airway and inserting the wire into the airway through the catheter; and
tracking a location includes tracking a length of the wire that extends into the airway out of the catheter.

17. The method of claim 16, further including inserting a tool into the airway over the wire.

18. The method of claim 17, wherein inserting the tool includes extending a portion of the wire through the tool.

19. The method of claim 16, wherein inserting a wire includes inserting a wire that includes three or more fiber optic cables with bragg gratings into the airway.

20. The method of claim 16, wherein tracking a location includes calculating a curvature of the wire in the airway.
